(19) United States
(12) Patent Application Publication
(10) Pub. No.: US 2026/0060656 A1
(43) Pub. Date: Mar. 5, 2026

(54) ULTRASOUND DIAGNOSIS APPARATUS, LESION DETECTION APPARATUS, AND LESION DETECTION METHOD

(71) Applicant: CANON MEDICAL SYSTEMS CORPORATION, Tochigi (JP)

(72) Inventors: Panjie GOU, Beijing (CN); Bing HAN, Beijing (CN); Shun ZHAO, Beijing (CN)

(73) Assignee: CANON MEDICAL SYSTEMS CORPORATION, Tochigi (JP)

(21) Appl. No.: 19/314,173

(22) Filed: Aug. 29, 2025

(30) Foreign Application Priority Data
Aug. 30, 2024 (CN) 202411209873.3

(51) Int. Cl. A61B 8/00; A61B 8/08

(52) U.S. Cl. CPC A61B 8/5223; A61B 8/085; A61B 8/461

(57) ABSTRACT

An ultrasound diagnosis apparatus and a lesion detection apparatus according to the embodiment include processing circuitry. The processing circuitry detects a lesion based on a detection threshold for detecting a lesion corresponding to a lesion type in each of a plurality of imaged frames, acquires detection results of the lesion in the plurality of frames, and determines a lesion type based on at least one of continuity and appearance frequency of the lesion type in the detection results of the plurality of frames, and updates the detection threshold according to the determined lesion type. The processing circuitry detects the lesion based on the updated detection threshold.

### CROSS-REFERENCE TO RELATED APPLICATIONS

This application is based upon and claims the benefit of priority from Chinese Patent Application No. 202411209873.3, filed on Aug. 30, 2024; the entire contents of all of which are incorporated herein by reference.

### FIELD

Embodiments disclosed in the present specification and the drawings relate to an ultrasound diagnosis apparatus, a lesion detection apparatus, and a lesion detection method.

### BACKGROUND

Image lesion detection technology is a local inspection means for detecting an organ or tissue to be measured in an image. The image lesion detection technology is widely applied to medical fields such as medical image analysis, auxiliary diagnosis, image management, and lesion detection and classification. Typical applications of the image lesion detection technology include colonoscopy, gastroscopy, ultrasonic endoscopy, and external ultrasonography.

In conventional image lesion detection technology, it is common to output a detection result to a user (doctor or technician) by displaying a detection frame in an image. However, in a case where the image lesion detection technology is used as an auxiliary diagnosis method, there are too many false detection frames, which may affect the user's observation and determination.

In addition, in the conventional image lesion detection technology, filtering is usually performed by setting a detection threshold to control the frequency and the number of false detection frames, and in this case, only a detection frame whose confidence exceeds the set detection threshold is output as the detection result. However, in a case where there are a plurality of kinds (types) of lesions in an image, the same detection threshold needs to be set for the plurality of types of lesions. Therefore, there is a possibility that the detection capability of a plurality of kinds of lesions is limited, making it impossible to reduce the number of false detection frames for a certain kind of lesion.

### BRIEF DESCRIPTION OF THE DRAWINGS

FIG. 1 is a block diagram illustrating an exemplary configuration of a lesion detection apparatus according to a first embodiment;

FIG. 2 is a schematic diagram illustrating detection results in a plurality of frames;

FIG. 3 is a schematic diagram illustrating grouping of the detection results in the plurality of frames;

FIG. 4 is a schematic diagram illustrating update of detection thresholds;

FIG. 5A is a schematic diagram illustrating display contents by a display unit;

FIG. 5B is a schematic diagram illustrating display contents by a display unit;

FIG. 6 is a flowchart of a lesion detection method according to the first embodiment;

FIG. 7A is a schematic diagram illustrating the detection result by the lesion detection apparatus according to the comparative example;

FIG. 7B is a schematic diagram illustrating the detection result by the lesion detection apparatus according to the first embodiment;

FIG. 8 is a block diagram illustrating an exemplary configuration of a lesion detection apparatus according to a second embodiment; and

FIG. 9 is a block diagram illustrating an exemplary configuration of an ultrasound diagnosis apparatus according to a third embodiment.

### DETAILED DESCRIPTION

One of problems to be solved by embodiments disclosed in the present specification and the drawings is to improve lesion detection accuracy when detecting a plurality of kinds of lesions. However, the problems to be solved by the embodiments disclosed in the present specification and the drawings are not limited to the above problem. A problem corresponding to each effect of each configuration described in the embodiments to be described later can also be positioned as another problem.

An ultrasound diagnosis apparatus according to an embodiment includes: a lesion detection unit that detects a lesion based on a detection threshold for detecting a lesion corresponding to a lesion type in each of a plurality of imaged frames; a frame result acquisition unit that acquires detection results of the lesion in the plurality of frames; and a threshold update unit that determines a lesion type based on at least one of continuity and appearance frequency of the lesion type in the detection results of the plurality of frames, and updates the detection threshold according to the determined lesion type, in which the lesion detection unit detects the lesion based on the updated detection threshold.

A lesion detection apparatus according to the present embodiment includes: a lesion detection unit that detects a lesion based on a detection threshold for detecting a lesion corresponding to a lesion type in each of a plurality of imaged frames; a frame result acquisition unit that acquires detection results of the lesion in the plurality of frames; and a threshold update unit that determines a lesion type based on at least one of continuity and appearance frequency of the lesion type in the detection results of the plurality of frames, and updates the detection threshold according to the determined lesion type, in which the lesion detection unit detects the lesion based on the updated detection threshold.

As a result, the ultrasound diagnosis apparatus and the lesion detection apparatus according to the present embodiment determine the lesion type based on the continuity and/or the appearance frequency of the lesion type in the detection results of the plurality of frames, and update the detection threshold according to the lesion type. Therefore, it is possible to update the detection threshold according to the lesion type in the plurality of frames, set the detection threshold of the lesion type in the plurality of frames separately from the detection threshold of a lesion type other than the lesion type in the plurality of frames without increasing the total number of detection frames, and improve the lesion detection accuracy. Here, a “lesion type in the current frame” is, for example, a lesion type whose possibility of existence in the current frame is equal to or greater than a predetermined threshold. In addition, a “lesion type other than the lesion type in the current frame” is, for example, a lesion type whose possibility of existence in the current frame is less than the predetermined threshold.

As compared with a case where the same or fixed detection threshold is set, it is possible to provide different detection thresholds by separating the lesion type in the plurality of frames from the lesion type other than the lesion type in the plurality of frames. Therefore, it is possible to reduce the possibility of detecting the lesion that does not exist in the plurality of frames, reduce the number of false detection frames, and further improve the lesion detection accuracy.

In the ultrasound diagnosis apparatus and the lesion detection apparatus according to the present embodiment, the frame result acquisition unit acquires detection results of the lesion in a plurality of previous frames before the current frame, the threshold update unit determines the lesion type in the current frame based on at least one of continuity and appearance frequency of the lesion type in the detection results of the plurality of previous frames, and updates the detection threshold according to the determined lesion type in the current frame, and the lesion detection unit detects the lesion in the current frame based on the updated detection threshold.

As a result, the ultrasound diagnosis apparatus and the lesion detection apparatus according to the present embodiment determine the lesion type in the current frame based on the continuity and/or the appearance frequency of the lesion type in the detection results of the previous frames, update the detection threshold according to the lesion type, and detect the lesion in the current frame based on the updated detection threshold. Therefore, it is possible to update the detection threshold according to the lesion type existing in the previous frames, detect the lesion in the current frame based on the updated detection threshold, and set the detection threshold of the lesion type existing in the current frame separately from the detection threshold of the lesion type not existing in the current frame without increasing the number of false detection frames, and thus it is possible to further improve the lesion detection accuracy. In addition, it is possible to reduce the possibility of detecting the lesion that does not exist in the current frame, reduce the number of false detection frames, and further improve the lesion detection accuracy.

In the ultrasound diagnosis apparatus and the lesion detection apparatus according to the present embodiment, the threshold update unit further determines the lesion type in the current frame based on the confidence of the lesion type in the detection results of the previous frames.

As a result, the ultrasound diagnosis apparatus and the lesion detection apparatus according to the present embodiment determine the lesion type in the current frame based on the confidence of the lesion type in the detection results of the previous frames. Therefore, it is possible to improve the accuracy of the lesion type determination in the current frame, and update the detection threshold according to the lesion type in the current frame, thereby further improving the lesion detection accuracy.

In the ultrasound diagnosis apparatus and the lesion detection apparatus according to the present embodiment, the threshold update unit updates the detection threshold so as to lower the detection threshold in a case where at least one of the continuity and the appearance frequency, and the confidence satisfy a preset condition.

In the ultrasound diagnosis apparatus and the lesion detection apparatus according to the present embodiment, the threshold update unit sets a lower limit to the updated detection threshold.

As a result, the ultrasound diagnosis apparatus and the lesion detection apparatus according to the present embodiment lower the detection threshold of the lesion type existing in the current frame and set the lower limit. Therefore, it is possible to avoid a reduction in the detection accuracy of the lesion existing in the current frame due to the threshold being too low, while increasing the possibility of detecting the lesion existing in the current frame, thereby further improving the lesion detection accuracy.

In the ultrasound diagnosis apparatus and the lesion detection apparatus according to the present embodiment, the threshold update unit updates the detection threshold so as to raise the detection threshold in the case where at least one of the continuity and the appearance frequency, and the confidence satisfy the preset condition.

In the ultrasound diagnosis apparatus and the lesion detection apparatus according to the present embodiment, the threshold update unit sets an upper limit to the updated detection threshold.

As a result, the ultrasound diagnosis apparatus and the lesion detection apparatus according to the present embodiment raise the detection threshold of the lesion type not existing in the current frame and set the upper limit. As a result, it is possible to avoid non-detection of some lesion in the current frame due to the threshold being too high, while reducing the possibility of detecting the lesion not existing in the current frame, thereby further improving the lesion detection accuracy.

In the ultrasound diagnosis apparatus and the lesion detection apparatus according to the present embodiment, the lesion detection unit causes a display to display a detection frame indicating the detected lesion in a case where the lesion is detected based on the updated detection threshold.

In the ultrasound diagnosis apparatus and the lesion detection apparatus according to the present embodiment, the lesion detection unit causes the display to display the detection frame so as to change the color of the detection frame according to the detected lesion type.

In the ultrasound diagnosis apparatus and the lesion detection apparatus according to the present embodiment, the lesion detection unit causes the display to display the detection frame together with the confidence of the detected lesion type.

In the ultrasound diagnosis apparatus and the lesion detection apparatus according to the present embodiment, the lesion detection unit causes the display to display information on update in a case where the detection threshold is updated.

As a result, the ultrasound diagnosis apparatus and the lesion detection apparatus according to the present embodiment cause the display to display the detection frame of the detected lesion, the confidence of the lesion type, the information on the update of the detection threshold, and the like. Therefore, it is possible to allow the user to easily check the update status of the detection threshold and the detection status of the lesion.

In the ultrasound diagnosis apparatus and the lesion detection apparatus according to the present embodiment, the threshold update unit updates the detection threshold in the current frame using a detection threshold output from a learned model by inputting the detection results in the previous frames to the learned model learned by a learning unit so as to output the detection threshold in the current frame based on the detection results in the previous frames.

As a result, the ultrasound diagnosis apparatus and the lesion detection apparatus according to the present embodiment update the detection threshold of the current frame by inputting the detection results in the previous frames to the learned model. Therefore, it is possible to appropriately update the detection threshold and further improve the lesion detection accuracy.

A lesion detection method according to the present embodiment includes: a lesion detection step of detecting a lesion based on a detection threshold for detecting a lesion corresponding to a lesion type in each of a plurality of imaged frames; a frame result acquisition step of acquiring detection results of the lesion in the plurality of frames; and a threshold update step of determining a lesion type based on at least one of continuity and appearance frequency of the lesion type in the detection results of the plurality of frames, and updating the detection threshold according to the determined lesion type, in which the lesion is detected based on the updated detection threshold in the lesion detection step.

Hereinafter, embodiments of the ultrasound diagnosis apparatus, the lesion detection apparatus, and the lesion detection method according to the embodiments will be described with reference to the drawings.

Note that, in the following description, the lesion detection apparatus and the lesion detection method according to the embodiments are used to detect lesions such as a hepatic cyst (Cyst), a hepatic hemangioma (Hema), a hepatic metastasis cancer (Meta), or a hepatocellular carcinoma (HCC) in a liver of a subject in an image (video) captured by ultrasonography performed by the ultrasound diagnosis apparatus. However, it goes without saying that the lesion detection apparatus and the lesion detection method according to the embodiments may be used to detect a lesion in an image captured by another medical imaging apparatus (another medical image diagnosis apparatus) or may be used to detect another lesion of an organ or tissue other than the liver of the subject.

### First Embodiment

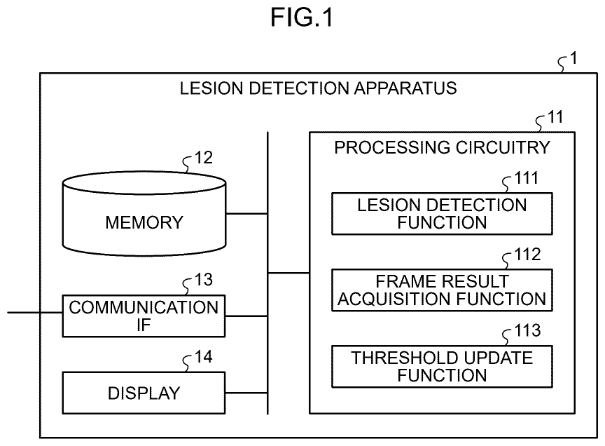

With reference to FIG. 1, an exemplary configuration of a lesion detection apparatus 1 according to a first embodiment will be described. FIG. 1 is a block diagram illustrating the exemplary configuration of the lesion detection apparatus 1 according to the first embodiment. As illustrated in FIG. 1, the lesion detection apparatus 1 includes a processing circuitry 11, a memory 12, a communication interface (IF) 13, and a display 14. The components are communicably connected to each other via a bus that is a common signal transmission path.

The memory 12 is a device that stores various data and information. The memory 12 may be a storage medium readable by a processor (e.g., a magnetic storage medium, an electromagnetic storage medium, an optical storage medium, or a semiconductor memory), or may be a drive device that reads and writes data and information from and to the storage medium. The memory 12 stores each computer program for causing the processing circuitry 11 to implement each functional unit.

The communication IF 13 is an interface for performing communication of various data and information between the components.

The processing circuitry 11 is a circuit that controls the entire operation of the lesion detection apparatus 1. The processing circuitry 11 includes at least one processor. The processor means a circuit such as a Central Processing Unit (CPU), a Graphics Processing Unit (GPU), an Application Specific Integrated Circuit (ASIC), and a programmable logic device (e.g., Simple Programmable Logic Device (SPLD), Complex Programmable Logic Device (CPLD), Field Programmable Gate Array (FPGA)). When the processor is the CPU, the CPU implements each function by reading and executing each program stored in the memory 12. When the processor is the ASIC, each function is directly incorporated into a circuit of the ASIC as a logic circuit. The processor may be configured as a single circuit, or may be configured by combining a plurality of independent circuits with each other. The processing circuitry 11 implements each function to be described later.

In the present embodiment, the processing circuitry 11 includes a lesion detection function 111, a frame result acquisition function 112, and a threshold update function 113. Hereinafter, each functional unit of the processing circuitry 11 will be described.

The lesion detection function 111 is a function for detecting the lesion in the image. The lesion detection function 111 detects the lesion based on the detection threshold for detecting the lesion corresponding to the lesion type in each of the plurality of imaged frames.

Here, in the following description, the “lesion type” includes, for example, the lesion type (lesion kind) such as hepatic cyst, hepatic hemangioma, hepatic metastasis cancer, and hepatocellular carcinoma. In addition, the “detection threshold” is, for example, a preset threshold corresponding to a lesion type, and is used to detect the corresponding type (kind) of lesion in the image. For example, the “detection threshold” is the “confidence” of the corresponding type of lesion detected in the image. The display 14 to be described later of the lesion detection apparatus 1 displays the detection frame whose confidence exceeds the detection threshold, and does not display the detection frame whose confidence is equal to or less than the detection threshold. For example, the lesion detection function 111 causes the display 14 to display the detection frame of the lesion type whose confidence exceeds the detection threshold, and does not cause the display 14 to display the detection frame of the lesion type whose confidence is equal to or less than the detection threshold. In this manner, the lesion detection function 111 detects the lesion type whose confidence exceeds the detection threshold and causes the display 14 to display the detection frame of the detected lesion type. Here, the “confidence” indicates a possibility that the detection result detected in the image is correct, and the higher the “confidence”, the higher the possibility that the detection result is correct. Note that the lesion detection function 111 can detect the lesion in the image, for example, using various known detection algorithms.

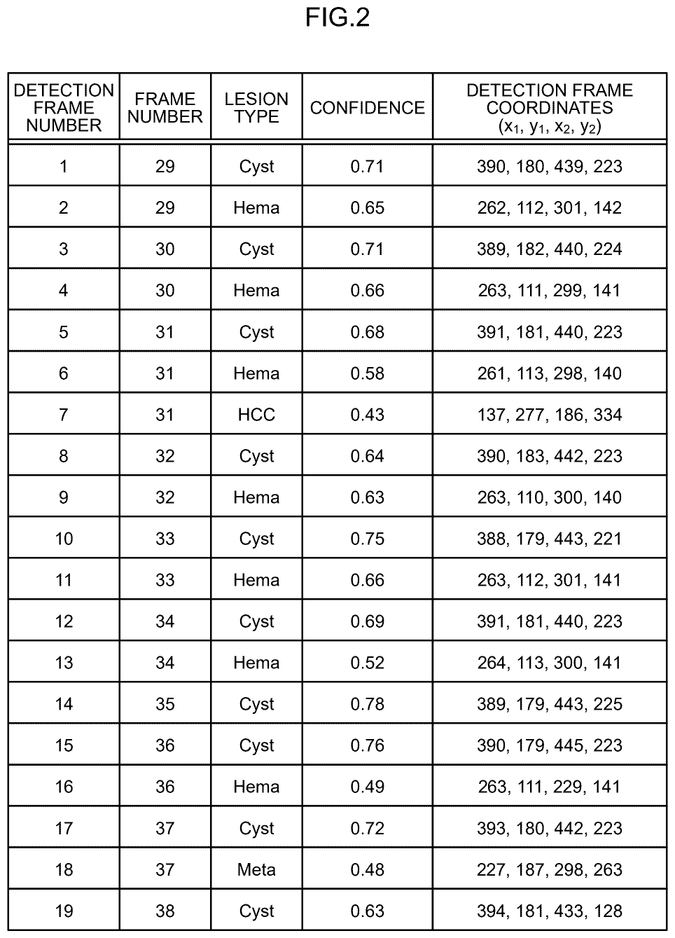

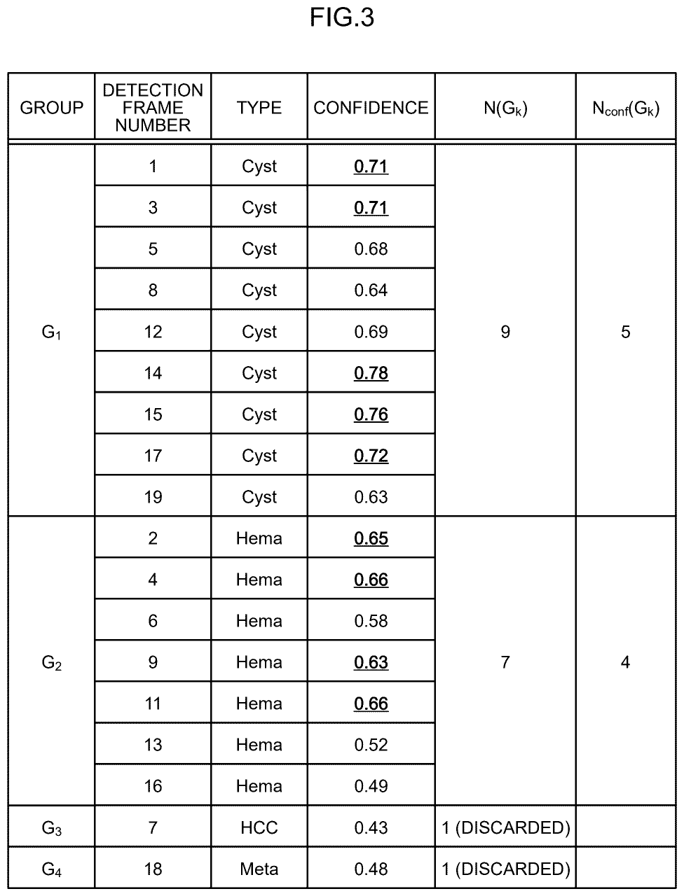

Detection results in a plurality of frames will be described with reference to FIG. 2, and the determination of the lesion type by the threshold update function 113 will be described with reference to FIG. 3. FIG. 2 is a schematic diagram illustrating the detection results in the plurality of frames according to the first embodiment. FIG. 3 is a schematic diagram illustrating grouping of the detection results in the plurality of frames according to the first embodiment. FIGS. 2 and 3 schematically illustrate the detection results in the 29th to 38th frames in the image. Note that the video includes one or more frames in the present specification. In addition, the frame refers to the image (image data). That is, one frame refers to one image.

Specifically, the lesion detection apparatus 1 acquires a video captured from a medical image generation apparatus (medical image diagnosis apparatus, modality) not illustrated in the drawing, the acquired video includes a plurality of frames, and the lesion detection function 111 detects a lesion included in each of the plurality of frames from each of the plurality of frames. In addition, the plurality of frames include the current frame and the previous frames. The current frame refers to, for example, the currently latest frame. The previous frames refer to one or more frames before the current frame.

The frame result acquisition function 112 is a function for acquiring the detection results in the plurality of frames. The frame result acquisition function 112 generates and acquires lesion detection results in the plurality of frames detected by the lesion detection function 111. Here, as illustrated in FIG. 2, the “detection results” include, for example, information in which a detection frame number, frame number, lesion type, confidence, and detection frame coordinates of the detection frames in the plurality of frames detected by the lesion detection function 111 are stored in association with each other. The frame result acquisition function 112 generates such detection results. The detection frame number is, for example, a number for uniquely identifying the detection frame. The frame number is, for example, a number for uniquely identifying the frame. The lesion type is, for example, information indicating the type (kind) of a lesion. The detection frame coordinates are a combination of the coordinates of diagonally positioned two vertices (x1, y1) and (x2, y2) out of the four vertices of the rectangular detection frame. The area of the region of the detection frame can be determined from the two vertices. The confidence indicates the possibility that the detection result detected in the image is correct, as described above. The higher the “confidence”, the higher the possibility that the detection result is correct.

The frame result acquisition function 112 transmits the detection results in the previous frames to the memory 12 and causes the memory 12 to store the detection results. The memory 12 stores, for example, a structure such as a queue or a list. In a case where the memory 12 stores the queue, the queue is used to store the detection results in the previous frames and the current frame. The length of the queue can be determined according to the frame rate of the image (video), and the higher the frame rate of the image, the longer the length of the queue. For example, in a case where the frame rate of the image is 22 frames/second, the length of the queue is 10.

The frame result acquisition function 112 may update the queue of the detection results by transmitting the detection result in the current frame to the memory 12 and adding the detection result in the current frame to the queue of the detection results stored in the memory 12.

Note that the lesion detection function 111 may execute the same processing as the processing executed by the frame result acquisition function 112. For example, the lesion detection function 111 may generate the detection result. In this case, the lesion detection function 111 or the frame result acquisition function 112 may transmit the generated detection result to the memory 12 and cause the memory 12 to store the detection result.

The threshold update function 113 is a function of updating the detection threshold. The threshold update function 113 determines the lesion type based on at least one of the continuity and the appearance frequency of the lesion type in the detection results of the plurality of frames, and updates the detection threshold according to the determined lesion type. Specifically, the threshold update function 113 determines the lesion type in the current frame (the lesion type considered to be highly likely to exist in the current frame) based on at least one of the continuity and the appearance frequency of the lesion type in the detection results of the plurality of previous frames, and updates the detection threshold according to the determined lesion type.

First, the threshold update function 113 groups the detection results in the queue of the detection results stored in the memory 12. The threshold update function 113 calculates an Intersection over Union (IoU) between two detection frames of the same lesion type in adjacent frames. The adjacent frames refer to, for example, two adjacent frames in a case where the frames are arranged in order of photographing (imaging) (chronological order). The Intersection over Union (IoU) is calculated, for example, by the following Formula 1.

$$IoU = \frac{Intersection ( Annotation , Detection )}{Union ( Annotation , Detection )} \qquad ( 1 )$$

Here, the IoU represents the Intersection over Union between two detection frames of the same lesion type in adjacent frames, Intersection (Annotation, Detection) represents an intersection region of the two detection frames (the area of a portion where the two detection frames (two regions of the two detection frames) overlap), and Union (Annotation, Detection) represents a union region of the two detection frames. Note that the union region of the two detection frames refers to, for example, the sum of the areas of the two regions of the two detection frames in a case where the two detection frames do not overlap, or the area of a region indicated by the two detection frames in a state where the two detection frames overlap in a case where the two detection frames overlap.

In addition, the threshold update function 113 presets a threshold T_{IoU} of the Intersection over Union (IoU). Note that the threshold T_{IoU} may be set in advance. For example, information indicating the threshold T_{IoU} is stored in advance in the memory 12, and the threshold update function 113 may acquire the information from the memory 12 and perform the following processing using the threshold T_{IoU} indicated by the acquired information. In the following description, the threshold T_{IoU} is 0.9, but the threshold T_{IoU} may be another appropriate value.

Next, the threshold update function 113 groups the detection results such that the Intersection over Union (IoU) between two detection frames of the same lesion type in adjacent frames in the same group exceeds the threshold T_{IoU}. For example, in a case where a plurality of frames of the same lesion type are arranged in order of photographing (imaging) (chronological order), the threshold update function 113 places into one group a plurality of detection results whose Intersection over Union (IoU) between two detection frames in two adjacent frames exceeds the threshold T_{IoU}. Note that, in a case where only one detection result is obtained for a certain lesion type from among the plurality of detection results, the threshold update function 113 sets this detection result as one group.

The detection frame of the lesion type “Cyst (hepatic cyst)” in FIG. 2 (detection frames 1, 3, 5, 8, 10, 12, 14, 15, 17, and 19) will be described as an example. In the case of the threshold T_{IoU}=0.9, an Intersection over Union (IoU_{1, 3}) between the detection frames 1 and 3, which have the same lesion type in the adjacent frames, an Intersection over Union (IoU_{3, 5}) between the detection frames 3 and 5, an Intersection over Union (IoU_{5, 8}) between the detection frames 5 and 8, an Intersection over Union (IoU_{8, 10}) between the detection frames 8 and 10, an Intersection over Union (IoU_{10, 12}) between the detection frames 10 and 12, an Intersection over Union (IoU_{12, 14}) between the detection frames 12 and 14, an Intersection over Union (IoU_{14, 15}) between the detection frames 14 and 15, an Intersection over Union (IoU_{15, 17}) between the detection frames 15 and 17, and an Intersection over Union (IoU_{17, 19}) between the detection frames 17 and 19 all exceed the threshold T_{IoU}, and thus, for example, as illustrated in FIG. 3, the detection frames 1, 3, 5, 8, 10, 12, 14, 15, 17, and 19 are placed into a group G₁. Note that the group is represented by “Gₖ”, and “k” is an identifier for identifying the group and is an integer of 1 or more.

Similarly, since Intersections over Union (IoUs) between two detection frames 2, 4, 6, 9, 11, 13, and 16 of the lesion type “Hema (hepatic hemangioma)” in adjacent frames all exceed the threshold T_{IoU}, the detection frames 2, 4, 6, 9, 11, 13, and 16 are placed into a group G₂.

In addition, since the queue of the detection results illustrated in FIG. 2 includes only one frame (frame 31) having the detection frame (detection frame 7) of the lesion type “HCC (hepatocellular carcinoma)”, the detection frame 7 is placed into a group G₃.

Similarly, since the queue of the detection results illustrated in FIG. 2 includes only one frame (frame 37) having the detection frame (detection frame 18) of the lesion type “Meta (hepatic metastasis cancer)”, the detection frame 18 is placed into a group G₄.

As a result, the threshold update function 113 groups the detection results in the queue of the detection results stored by the memory 12 into groups G₁, G₂, G₃, and G₄ based on the Intersection over Union (IoU) between two detection frames of the same lesion type in adjacent frames.

Here, the Intersection over Union (IoU) reflects “continuity” between two detection frames of a certain lesion type. That is, the Intersection over Union (IoU) is information indicating such “continuity”. In a case where the Intersection over Union (IoU) between two detection frames of the certain lesion type in adjacent frames exceeds the threshold Troy, the threshold update function 113 places the detection frames into one group because the continuity between the detection frames for the lesion type is considered to be high. Note that, in the above description, the threshold update function 113 calculates the “Intersection over Union (IoU)” between two detection frames as the information indicating “continuity” and groups the detection results. However, the threshold update function may group the detection results by using various types of information such as other parameters or indexes that can reflect the continuity between two detection frames of a certain lesion type in adjacent frames.

Next, the threshold update function 113 counts the number N (Gₖ) of detection frames in each group Gₖ, and presets a threshold T_{N} of the number N (Gₖ) of detection frames. Note that the threshold T_{N} may be set in advance. At this time, as illustrated in FIG. 3, the number N (G₁) of detection frames in the group G₁ is 9, the number N (G₁) of detection frames in the group G₂ is 7, the number N (G₃) of detection frames in the group G₃ is 1, and the number N (G₄) of detection frames in the group G₄ is 1. Note that, in the following description, the threshold T_{N} is 5, but the threshold T_{N} may be another appropriate value.

The threshold update function 113 compares the number N (Gₖ) of detection frames in each group Gₖ with the threshold T_{N}, and leaves the group Gₖ whose number N (Gₖ) of detection frames exceeds the threshold T_{N} while discarding the group whose number N (Gₖ) of detection frames does not exceed the threshold T_{N}. For example, in the example illustrated in FIG. 3, since the number N (G₁) of detection frames in the group G₁ and the number N (G₂) of detection frames in the group G₂ exceed the threshold T_{N}, the detection frames in the group G₁ and the group G₂ are left, but the detection frames in the group G₃ and the group G₄ are discarded.

Here, the number N (Gₖ) of detection frames in each group Gₖ reflects the “appearance frequency” of a detection frame of a certain lesion type. That is, the number N (Gₖ) of detection frames is information indicating the “appearance frequency” of the detection frame of the certain lesion type. In a case where the number N (Gₖ) of detection frames in a certain group Gₖ exceeds the threshold T_{N}, the detection frames in the group Gₖ are left because the appearance frequency of the detection frames for the lesion type corresponding to the group Gₖ is considered to be high.

Next, the threshold update function 113 determines the lesion type corresponding to the left group as the lesion type in the current frame. For example, the threshold update function 113 determines the lesion type Cyst corresponding to the left group G₁ and the lesion type Hema corresponding to the group G₂ as the lesion types in the current frame. Here, a “lesion type in the current frame” is, for example, a lesion type whose possibility of existence in the current frame is equal to or greater than a predetermined threshold. In addition, a “lesion type other than the lesion type in the current frame” is, for example, a lesion type whose possibility of existence in the current frame is less than the predetermined threshold.

As a result, the lesion detection apparatus 1 according to the present embodiment determines the lesion type in the current frame based on the continuity and the appearance frequency of the lesion type in the detection results of the previous frames, and can improve the accuracy of the determination of the lesion type in the current frame.

Note that, in the above description, an example has been described in which the threshold update function 113 places the detection results into the plurality of groups Gr based on the Intersection over Union (IoU) (continuity), leaves some of the plurality of groups Gr based on the number N (Gₖ) (appearance frequency) of detection frames, and then determines the lesion types corresponding to the left groups as the lesion types in the current frame. However, the threshold update function 113 may determine the lesion type based on either the continuity or the appearance frequency of the lesion type in the detection result.

For example, the threshold update function 113 may place the detection results into the plurality of groups based on the Intersection over Union (IoU) (continuity), and then determine the lesion type corresponding to each of all the groups as the lesion type in the current frame. In addition, the threshold update function 113 may determine the lesion type in the current frame based on the number (appearance frequency) of detection frames in the detection results without grouping. For example, the threshold update function 113 compares the number (appearance frequency) of detection frames in the detection results with the predetermined threshold for each lesion type without grouping. Then, the threshold update function 113 determines a lesion type whose number of detection frames exceeds the predetermined threshold among all the lesion types as the lesion type in the current frame.

Furthermore, the threshold update function 113 may further determine the lesion type in the current frame based on the confidence of the lesion type in the detection results of the previous frames.

Specifically, the threshold update function 113 presets a threshold T_{conf} of the confidence and a threshold I_{Nconf} of a number N_{conf} (Gₖ) of detection frames. The threshold update function 113 sets the threshold T_{conf} for each lesion type. Note that the threshold T_{conf} and the threshold T_{Nconf} may be preset. For example, the threshold T_{conf} may be set for each lesion type. In the following description, the threshold I_{conf} of the lesion type Cyst is 0.708, the threshold T_{conf} of the lesion type Hema is 0.615, and the threshold I_{Nconf} is 3.

Next, the threshold update function 113 counts the number N_{conf} (Gₖ) of detection frames whose confidence exceeds the threshold T_{conf}. At this time, as indicated by a bold underline in FIG. 3, the number of detection frames N_{conf} (G₁) in the group G₁ whose confidence exceeds the threshold T_{conf} is 5 (detection frames 1, 3, 14, 15, and 17), and the number of detection frames N_{conf} (G₂) in the group G₂ whose confidence exceeds the threshold T_{conf} is 4 (detection frames 2, 4, 9, and 11).

Next, the threshold update function 113 compares the number N_{conf} (Gₖ) of detection frames whose confidence exceeds the threshold T_{conf} in each group with the threshold I_{Nconf}, and determines the lesion type corresponding to the group whose number N_{conf} (Gₖ) of detection frames exceeds the threshold T_{Nconf} as the lesion type in the current frame.

Here, the number of detection frames N_{conf} (G₁) in the group G₁ whose confidence exceeds the threshold T_{conf} and the number of detection frames N_{conf} (G₂) in the group G₂ whose confidence exceeds the threshold T_{conf} exceed the threshold T_{Nconf}. Therefore, the lesion type Cyst corresponding to the group G₁ and the lesion type Hema corresponding to the group G₂ are determined as the lesion type in the current frame.

As a result, the lesion detection apparatus 1 according to the present embodiment can also determine the lesion type in the current frame based on the confidence of the lesion type in the detection results of the previous frames, and can improve the accuracy of the determination of the lesion type in the current frame.

In addition, in lesion detection from images, detection frames with a correctly detected lesion often appear continuously or at regular intervals, and have high confidence. Therefore, the lesion detection apparatus 1 according to the present embodiment determines the lesion type in the current frame based on the continuity and/or the appearance frequency, and the confidence of the lesion type in the detection results of the previous frames, thereby making it possible to improve the accuracy of the determination of the lesion type in the current frame.

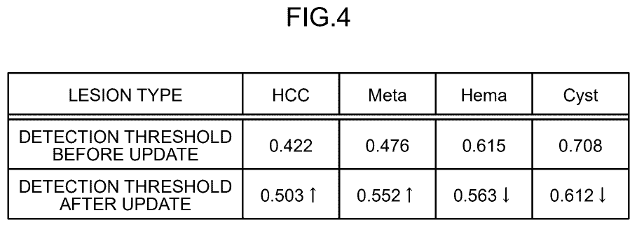

With reference to FIG. 4, the update of the detection threshold will be described. FIG. 4 is a schematic diagram illustrating the update of the detection thresholds according to the first embodiment.

The threshold update function 113 updates the detection threshold based on the determined lesion type in the current frame.

Specifically, the threshold update function 113 may update, for the determined lesion type in the current frame, the detection threshold of the lesion type so as to lower the detection threshold. For example, the threshold update function 113 may update, for the determined lesion type in the current frame, the detection threshold of the lesion type so as to lower the detection threshold by a predetermined value. In addition, the threshold update function 113 may update, for the lesion type not existing in the current frame, the detection threshold of the lesion type so as to raise the detection threshold. For example, the threshold update function 113 may update, for the lesion type not existing in the current frame, the detection threshold of the lesion type so as to raise the detection threshold by a predetermined value.

For example, as illustrated in FIG. 4, in a case where it is determined that the lesion type in the current frame is Cyst and Hema, and that the lesion types HCC and Meta do not exist in the current frame, the threshold update function 113 updates the detection thresholds so as to lower the detection threshold corresponding to the lesion type Cyst and the detection threshold corresponding to the lesion type Hema, while raising the detection threshold corresponding to the lesion type HCC and the detection threshold corresponding to the lesion type Meta.

Next, the lesion detection function 111 detects the lesion in the current frame based on the updated detection threshold. For example, the lesion detection function 111 causes the display 14 to display the detection frame of the lesion type whose confidence exceeds the updated detection threshold, and does not cause the display 14 to display the detection frame of the lesion type whose confidence is equal to or less than the updated detection threshold.

As a result, the lesion detection apparatus 1 according to the present embodiment can update the detection threshold according to the lesion type in the current frame, and lower the detection threshold corresponding to the lesion type existing in the current frame so as to increase the possibility of detecting the type of lesion, while raising the detection threshold corresponding to the lesion type not existing in the current frame so as to reduce the possibility of detecting the type of lesion not existing in the current frame. Therefore, the lesion detection apparatus 1 according to the present embodiment can update the detection threshold corresponding to the lesion type existing in the current frame without increasing the number of false detection frames, and improve the lesion detection accuracy.

In addition, the threshold update function 113 may set an upper limit or a lower limit to the updated detection threshold. Note that the upper limit or the lower limit may be set in advance to the updated detection threshold. For example, the threshold update function 113 sets the upper limit to the updated detection threshold when raising the detection threshold, and sets the lower limit to the updated detection threshold when lowering the detection threshold.

The lesion type HCC and the lesion type Meta will be described as an example. To avoid a reduction in the possibility of detecting the malignant disease such as a hepatic metastasis cancer (Meta) or a hepatocellular carcinoma (HCC) in the current frame due to the detection threshold being too high, the threshold update function 113 sets the upper limits to the detection threshold corresponding to the lesion type HCC and the detection threshold corresponding to the lesion type Meta to prevent the updated detection threshold from being too high.

In addition, to avoid a reduction in the detection accuracy in the current frame due to the detection threshold being too low, the threshold update function 113 may set the lower limits to the detection threshold corresponding to the lesion type Hema and the detection threshold corresponding to the lesion type Cyst to prevent the updated detection threshold from being too low.

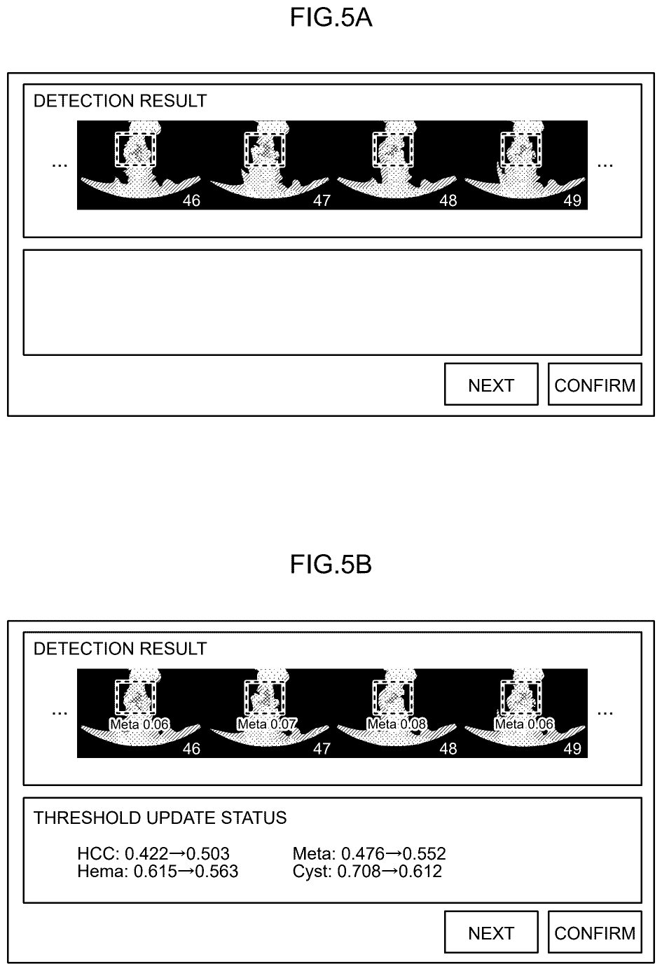

With reference to FIGS. 5A and 5B, display contents of the display 14 will be described. FIGS. 5A and 5B are schematic diagrams illustrating an example of display contents of the display 14 according to the first embodiment. The display 14 displays various images and various types of information under the control of the lesion detection function 111. That is, the lesion detection function 111 causes the display 14 to display the various images and the various types of information.

As illustrated in FIG. 5A, in a case where the lesion is detected based on the detection threshold updated by the lesion detection function 111, the display 14 displays the detection frame (broken line frame in FIG. 5A) indicating the detected lesion. The display 14 may display the detection frame so as to change the color of the detection frame according to the detected lesion type (not illustrated). For example, the lesion detection function 111 changes the color of the detection frame to be displayed on the display 14 to blue when the detected lesion type is Cyst, and changes the color of the detection frame to be displayed on the display 14 to green when the detected lesion type is Hema. The lesion detection function 111 may change the color of the detection frame to be displayed on the display 14 to other colors.

In addition, as illustrated in FIG. 5B, the lesion detection function 111 causes the display 14 to display the detection frame together with the confidence of the detected lesion type. The lesion detection function 111 may cause the display 14 to display the information on update (“threshold update status” in FIG. 5B) in a case where the detection threshold is updated.

As a result, the lesion detection apparatus 1 causes the display 14 to display the detection frame of the detected lesion, the confidence of the lesion type, the information on the update of the detection threshold, and the like. Therefore, it is possible to allow the user to easily check the update status of the detection threshold and the detection status of the lesion.

### Flow of First Embodiment

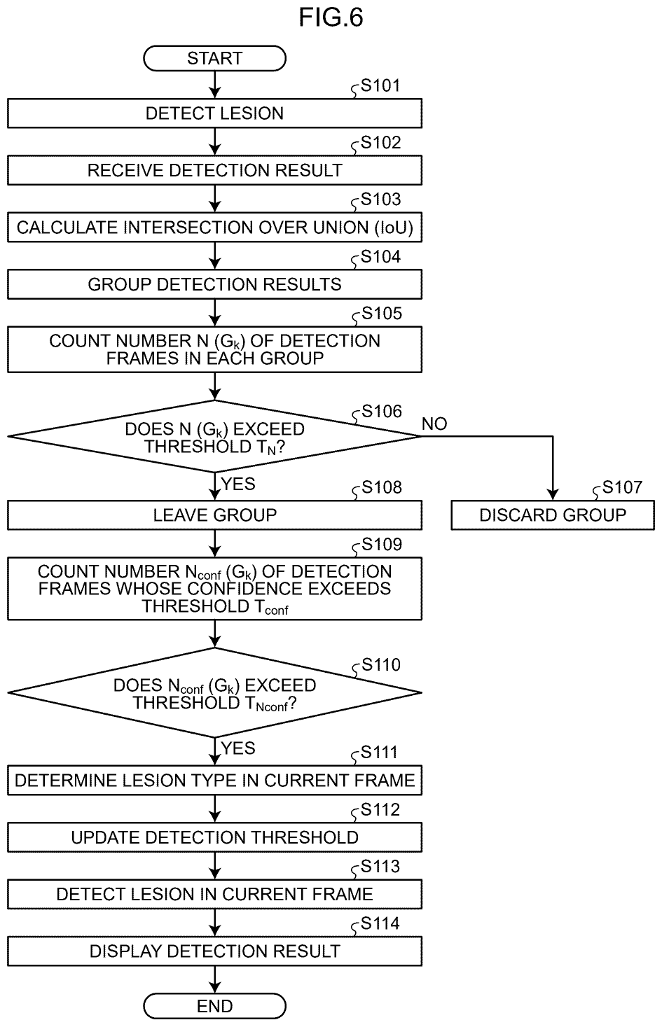

Hereinafter, a flow of the lesion detection method according to the first embodiment (a flow of lesion detection processing according to the first embodiment) will be described with reference to FIG. 6. FIG. 6 is a flowchart of a lesion detection method according to the first embodiment.

In step S101, the lesion detection apparatus 1 acquires the captured video, and the lesion detection function 111 detects the lesion in each frame of the image. Next, the processing proceeds to step S102.

In step S102, the frame result acquisition function 112 receives the detection results in the previous frames detected by the lesion detection function 111. In addition, the frame result acquisition function 112 transmits the detection results in the previous frames to the memory 12 and causes the memory 12 to store the detection results in the queue form. Next, the processing proceeds to step S103.

In step S103, the threshold update function 113 calculates the Intersection over Union (IoU) between two detection frames of the same lesion type in adjacent frames based on the detection results stored in the memory 12. Next, the processing proceeds to step S104.

In step S104, the threshold update function 113 groups the detection results such that the Intersection over Union (IoU) calculated in step S103 between the two detection frames of the same lesion type in the adjacent frames in the same group exceeds the preset threshold T_{IoU}. Next, the processing proceeds to step S105.

In step S105, the threshold update function 113 counts the number N (Gₖ) of detection frames in each group obtained by the grouping in step S104.

Here, the processing of steps S106 to S113 to be described below is executed for each group. For example, between the processing of step S105 and the processing of step S106, the threshold update function 113 executes processing of determining whether or not there is an unselected group among one or more groups obtained by the grouping in step S104. Then, when it is determined that there is an unselected group, the threshold update function 113 selects one group from among the unselected groups. Then, the selected group is subjected to the processing of steps S106 to S113. When it is determined that there is no unselected group, the threshold update function 113 proceeds to step S114.

In step S106, the threshold update function 113 compares the number N (Gₖ) of detection frames in the selected group with the threshold T_{N}, and proceeds to step S107 when the number N (Gₖ) of detection frames in the selected group does not exceed the threshold T_{N}, and proceeds to step S108 when the number N (Gₖ) of detection frames in the selected group exceeds the threshold T_{N}.

In step S107, the threshold update function 113 discards the group whose number N (Gₖ) of detection frames does not exceed the threshold T_{N}.

In step S108, the threshold update function 113 leaves the group whose number N (Gₖ) of detection frames exceeds the threshold T_{N}. Next, the processing proceeds to step S109.

In step S109, the threshold update function 113 counts the number N_{conf} (Gₖ) of detection frames whose confidence exceeds the threshold T_{conf}. Next, the processing proceeds to step S110.

In step S110, the threshold update function 113 compares the number N_{conf} (Gₖ) of detection frames whose confidence exceeds the threshold T_{conf} in the selected group with the threshold I_{Nconf}, and when the number N_{conf} (Gₖ) of detection frames exceeds the threshold I_{Nconf}, the processing proceeds to step S111. When the number of detection frames N_{conf} (Gₖ) does not exceed the threshold I_{Nconf}, the processing after step S111 is not executed.

In step S111, the threshold update function 113 determines the lesion type corresponding to the selected group as the lesion type in the current frame. Next, the processing proceeds to step S112.

In step S112, the threshold update function 113 updates the detection threshold according to the lesion type in the current frame determined in step S111. For example, the threshold update function 113 updates the detection threshold so as to lower the detection threshold corresponding to the lesion type in the current frame and raise the detection threshold corresponding to the lesion type other than the lesion type in the current frame. Next, the processing proceeds to step S113.

In step S113, the lesion detection function 111 detects the lesion in the current frame based on the detection threshold updated in step S112.

In step S114, the lesion detection function 111 causes the display 14 to display the lesion detected in step S113 for all the groups whose number N_{conf} (Gₖ) of detection frames is determined to exceed the threshold T_{Nconf} in step S110. The display 14 may display the detection frame of the detected lesion, the confidence of the lesion type, the information on the update of the detection threshold, and the like under the control of the lesion detection function 111. This ends the flow illustrated in FIG. 6.

### COMPARATIVE EXAMPLE

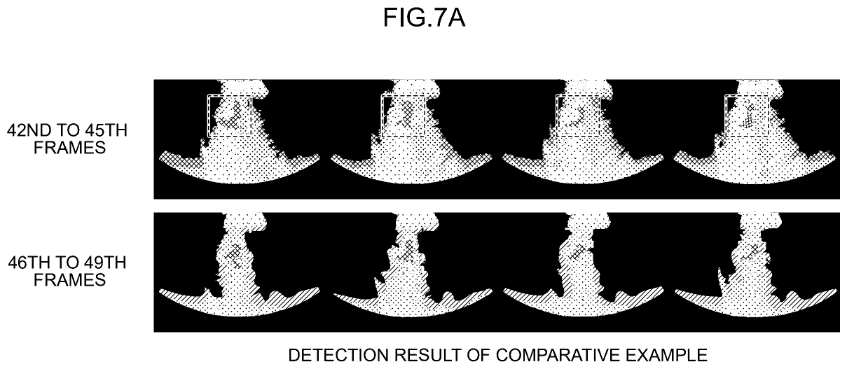

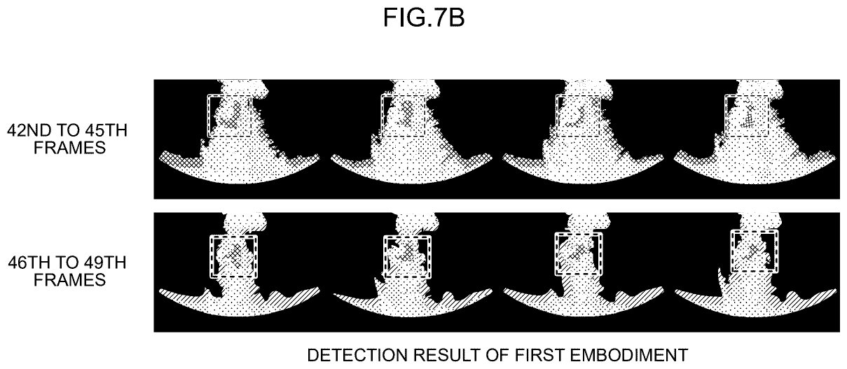

With reference to FIGS. 7A and 7B, a comparison between a detection result by a lesion detection apparatus according to a comparative example and the detection result by the lesion detection apparatus 1 according to the first embodiment will be described. FIG. 7A is a schematic diagram illustrating the detection result by the lesion detection apparatus according to the comparative example. FIG. 7B is a schematic diagram illustrating the detection result by the lesion detection apparatus 1 according to the first embodiment. FIG. 7A illustrates the detection result by the lesion detection apparatus according to the comparative example, and FIG. 7B is the detection result by the lesion detection apparatus 1 according to the first embodiment.

Note that, in the following description, an example will be described in which the lesion detection apparatus according to the comparative example and the lesion detection apparatus according to the first embodiment detect a hepatic metastasis cancer (Meta) in a video.

As illustrated in FIG. 7A and FIG. 7B, a lesion region of the hepatic metastasis cancer in 42nd to 45th frames in the video has high contrast and clear edges. Therefore, both the lesion detection apparatus according to the comparative example and the lesion detection apparatus 1 according to the first embodiment can detect the detection result of the hepatic metastasis cancer (Meta) with high confidence in the 42nd to 45th frames and display the detection frame.

However, a lesion region of the hepatic metastasis cancer in 46th to 49th frames in the video has poor contrast and blurred edges. Therefore, the lesion detection apparatus according to the comparative example cannot detect the hepatic metastasis cancer (Meta) in the 46th to 49th frames and cannot display the detection frame. On the other hand, the lesion detection apparatus 1 according to the first embodiment can detect the hepatic metastasis cancer (Meta) in the 46th to 49th frames and display the detection frame as in the 42nd to 45th frames.

This is because, in the lesion detection apparatus according to the comparative example, the detection threshold is usually set to a fixed value, and for example, the detection threshold corresponding to the lesion type Meta is set to 0.076. At this time, for the frames with high contrast (42nd to 45th frames), a detection result with high confidence can be detected and the detection frame can be displayed, but for the frames with low contrast (46th to 49th frames), a detection result higher than the set detection threshold cannot be detected and none of the detection frames is displayed.

On the other hand, in the lesion detection apparatus 1 according to the first embodiment, the threshold update function 113 determines the lesion type in the current frame based on the detection results of the previous frames, and updates the detection threshold according to the determined lesion type in the current frame. Therefore, in the current frame, the detection threshold can be appropriately updated according to the lesion type.

As a result, for example, in a case where the contrast of the 46th to 49th frames is low, the threshold update function 113 can update the detection threshold by determining the lesion type in the 46th to 49th frames from the detection results in the previous frames before the 46th to 49th frames.

For example, in a case where it is determined that the lesion type in the 46th to 49th frames is the hepatic metastasis cancer (Meta), the threshold update function 113 can update the detection threshold by appropriately lowering the detection threshold corresponding to the lesion type Meta to 0.035. In this case, the threshold update function 113 can detect the detection result exceeding the updated detection threshold in the 46th to 49th frames and display the detection frame.

### Second Embodiment

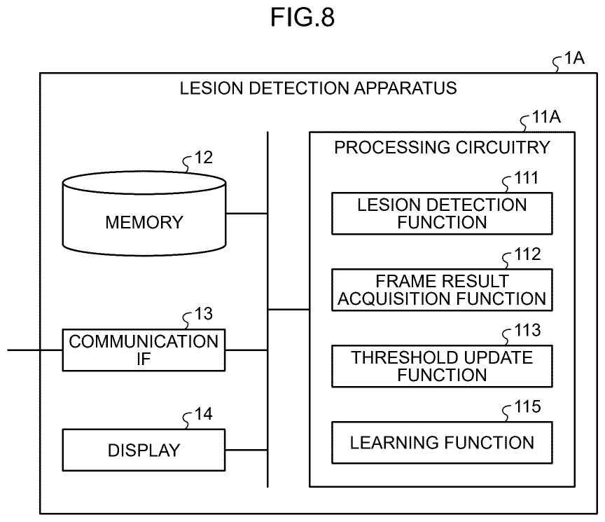

With reference to FIG. 8, an exemplary configuration of a lesion detection apparatus 1A according to a second embodiment will be described. FIG. 8 is a block diagram illustrating the exemplary configuration of the lesion detection apparatus 1A according to the second embodiment. Hereinafter, only differences between the lesion detection apparatus 1A according to the second embodiment and the lesion detection apparatus 1 according to the first embodiment will be described, and description of common points may be omitted.

As illustrated in FIG. 8, processing circuitry 11A of the lesion detection apparatus 1A further includes a learning function 115.

The learning function 115 is a functional unit that causes a learned model to learn. The learned model is stored in the memory 12.

The learning function 115 generates the learned model by causing the model to learn so as to set (output) the detection threshold in the current frame based on the detection results in the previous frames. Then, the learning function 115 causes the memory 12 to store the generated learned model. Then, the threshold update function 113 updates the detection threshold in the current frame by inputting the detection results in the previous frames to the learned model stored in the memory 12.

Specifically, for example, the threshold update function 113 inputs the lesion type, the position (detection frame coordinates), and the confidence corresponding to the detection frames in the previous frames to the learned model, and causes the learned model to output the detection threshold corresponding to each lesion type. Then, the threshold update function 113 updates the detection threshold corresponding to the corresponding lesion type by setting the detection threshold output from the learned model as the detection threshold corresponding to the corresponding lesion type.

In addition, in the training stage (learning stage) of the learned model, it is possible to improve the detection rate (the rate at which the detection frame labeled manually matches the detection frame output by the learned model) using a manually labeled detection frame as a teacher, and cause the learned model to learn by setting the detection threshold target of reducing the false detection rate (the rate at which the manually labeled detection frame does not match the detection frame output by the learned model).

As a result, the lesion detection apparatus 1A according to the second embodiment updates the detection threshold of the current frame by inputting the detection results in the previous frames to the learned model. Therefore, it is possible to appropriately update the detection threshold and further improve the lesion detection accuracy.

### Third Embodiment

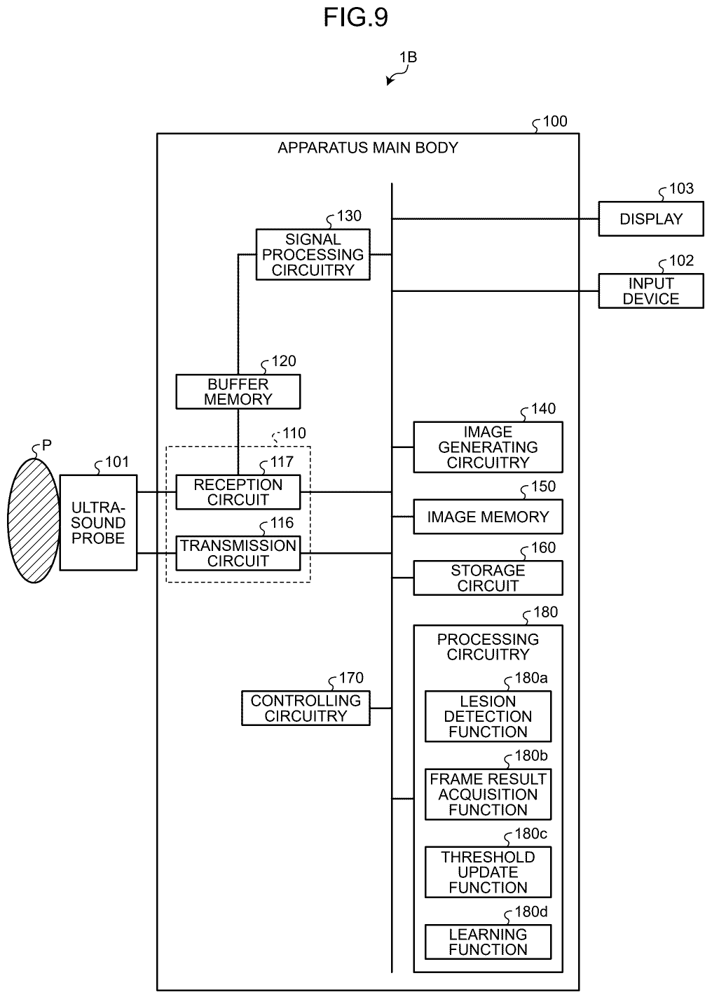

FIG. 9 is a block diagram illustrating an exemplary configuration of an ultrasound diagnosis apparatus 1B according to a third embodiment. As illustrated in FIG. 9, the ultrasound diagnosis apparatus 1B according to the first embodiment includes an apparatus main body 100, an ultrasound probe 101, an input device 102, and a display 103.

For example, the ultrasound probe 101 includes a plurality of elements (piezoelectric transducer elements or piezoelectric elements). The plurality of elements are configured to generate an ultrasound wave on the basis of a drive signal supplied from a transmission circuit 116 of a transmission and reception circuit 110 included in the apparatus main body 100. More specifically, as a result of the transmission circuit 116 applying voltage (transmission drive voltage) thereto, the plurality of elements are configured to generate the ultrasound wave having a waveform corresponding to the transmission drive voltage. The waveform of the transmission drive voltage indicated by the drive signal is the waveform of the voltage applied to the plurality of elements. In other words, the ultrasound probe 101 is configured to transmit the ultrasound wave corresponding to the magnitude of the applied transmission drive voltage. Further, the ultrasound probe 101 is configured to receive a reflected wave arriving from an examined subject (hereinafter, “patient”) P, to convert the reflected wave into a reception signal (a reflected-wave signal) being an electrical signal, and to output the reception signal to the apparatus main body 100. Further, the ultrasound probe 101 includes, for example, a matching layer provided for the elements, a backing member configured to prevent ultrasound waves from propagating rearward from the elements, and the like. In this situation, the ultrasound probe 101 is detachably connected to the apparatus main body 100.

When the ultrasound wave is transmitted from the ultrasound probe 101 to the patient P, the transmitted ultrasound wave is repeatedly reflected on a surface of discontinuity of acoustic impedances at a tissue in the body of the patient P, so as to be received as the reflected wave by the plurality of elements included in the ultrasound probe 101. The amplitude of the received reflected wave is dependent on the difference between the acoustic impedances on the surface of discontinuity on which the ultrasound wave is reflected. Further, when a transmitted ultrasound pulse is reflected on the surface of a moving blood flow, a cardiac wall, or the like, the reflected wave is, due to the Doppler effect, subject to a frequency shift, depending on a velocity component of the moving members with respect to the ultrasound wave transmission direction. Further, the ultrasound probe 101 is configured to output the reception signal to reception circuit 117 (explained later) of the transmission and reception circuit 110.

The ultrasound probe 101 is provided so as to be attachable to and detachable from the apparatus main body 100. When a two-dimensional region in the patient P is to be scanned (a two-dimensional scan), for example, an operator connects, as the ultrasound probe 101, a one-dimensional (1D) array probe in which the plurality of elements are arranged in a single row, to the apparatus main body 100. Examples of different types of the 1D array probe include linear ultrasound probes, convex ultrasound probes, and sector ultrasound probes. Further, when a three-dimensional region in the patient P is to be scanned (a three-dimensional scan), for example, the operator connects, as the ultrasound probe 101, a mechanical four-dimensional (4D) probe or a two-dimensional (2D) array probe, to the apparatus main body 100. The mechanical 4D probe is capable of performing a two-dimensional scan by using the plurality of elements arranged in a single row like in the 1D array probe and is also capable of performing a three-dimensional scan by swinging the plurality of elements at a predetermined angle (a swing angle). Further, the 2D array probe is capable of performing a three-dimensional scan by using the plurality of elements arranged in a matrix formation and is also capable of performing a two-dimensional scan by transmitting an ultrasound wave in a converged manner.

The input device 102 is realized, for example, by using input means such as a mouse, a keyboard, a button, a panel switch, a touch command screen, a foot switch, a trackball, a joystick, and/or the like. The input device 102 is configured to receive various types of setting requests from the operator of the ultrasound diagnosis apparatus 1B and to transfer the received various types of setting requests to the apparatus main body 100.

The display 103 is configured, for example, to display a Graphical User Interface (GUI) used by the operator of the ultrasound diagnosis apparatus 1B for inputting the various types of setting requests via the input device 102 and to display an ultrasound image based on ultrasound image data generated in the apparatus main body 100, and the like. The display 103 is realized by using a liquid crystal monitor, a Cathode Ray Tube (CRT) monitor, or the like.

The apparatus main body 100 is configured to generate the ultrasound image data on the basis of the reception signal transmitted thereto from the ultrasound probe 101. In this situation, the ultrasound image data is an example of image data. The apparatus main body 100 is capable of generating two-dimensional ultrasound image data on the basis of the reception signal corresponding to a two-dimensional region of the patient P and having been transmitted thereto from the ultrasound probe 101. Further, the apparatus main body 100 is capable of generating three-dimensional ultrasound image data on the basis of the reception signal corresponding to a three-dimensional region of the patient P and having been transmitted thereto from the ultrasound probe 101. As illustrated in FIG. 1, the apparatus main body 100 includes the transmission and reception circuit 110, a buffer memory 120, signal processing circuitry 130, image generating circuitry 140, an image memory 150, storage circuit 160, controlling circuitry 170, and processing circuitry 180.

The transmission and reception circuit 110 is configured, under control of the controlling circuitry 170, to cause the ultrasound probe 101 to transmit the ultrasound wave and to cause the ultrasound probe 101 to receive the reflected wave of the ultrasound wave. In other words, the transmission and reception circuit 110 is configured to perform the scan via the ultrasound probe 101. The transmission and reception circuit 110 is an example of a transmission and reception unit. The transmission and reception circuit 110 includes the transmission circuit 116 and the reception circuit 117. The transmission circuit 116 is an example of a transmission unit, whereas the reception circuit 117 is an example of a reception unit.

Under the control of the controlling circuitry 170, the transmission circuit 116 is configured to cause the ultrasound wave to be transmitted from the ultrasound probe 101. The transmission circuit 116 includes rate pulser generating circuit, transmission delay circuit, and a transmission pulser. The transmission circuit 116 is configured to supply the drive signal to the ultrasound probe 101. When a two-dimensional region in the patient P is to be scanned, the transmission circuit 116 is configured to cause the ultrasound probe 101 to transmit an ultrasound beam for scanning the two-dimensional region. In contrast, when a three-dimensional region in the patient P is to be scanned, the transmission circuit 116 is configured to cause the ultrasound probe 101 to transmit an ultrasound beam for scanning the three-dimensional region.

Under control of the controlling circuitry 170, the rate pulser generating circuit is configured to repeatedly generate a rate pulse for forming a transmission ultrasound wave (a transmission beam) at a predetermined rate frequency (a Pulse Repetition Frequency [PRF]). As a result of the rate pulse being routed through the transmission delay circuit, voltage is applied to the transmission pulser while having mutually-different transmission delay time periods. For example, the transmission delay circuit is configured to apply the transmission delay time period that corresponds to each of the elements and is required to converge the ultrasound wave generated from the ultrasound probe 101 into the form of a beam and to determine transmission directionality, to the rate pulses generated by the rate pulser generating circuit. The transmission pulser is configured to supply the drive signal (a drive pulse) to the ultrasound probe 101 with timing based on the rate pulses. In other words, the transmission pulser is configured to apply the voltage (the transmission drive voltage) having the waveform indicated by the drive signal to the ultrasound probe 101, with the timing based on the rate pulses. In this situation, the transmission delay circuit is configured to arbitrarily adjust the transmission direction of the ultrasound wave arriving from the surfaces of the elements, by varying the transmission delay time periods applied to the rate pulses.

The drive pulse travels from the transmission pulser and reaches the elements provided in the ultrasound probe 101 via a cable and is subsequently converted at the elements from an electrical signal into mechanical vibration. In other words, as a result of the voltage being applied to the elements, the elements are configured to mechanically vibrate. The ultrasound wave generated by the mechanical vibration is transmitted to the inside of the patient's body (the inside of the patient P). In this situation, the ultrasound waves having the mutually-different transmission delay time periods in correspondence with the elements are converged so as to propagate in a predetermined direction.

Further, under the control of the controlling circuitry 170, the transmission circuit 116 has a function capable of instantaneously changing the transmission frequency, the transmission drive voltage, and the like for executing a predetermined scan sequence. In particular, the capability to change the transmission drive voltage is realized by a transmission circuit of a linear amplifier type capable of instantaneously switching the value of the transmission drive voltage or a mechanism configured to electrically switch between a plurality of power source units.

The reflected wave of the ultrasound wave transmitted by the ultrasound probe 101 reaches the elements provided inside the ultrasound probe 101 and is subsequently converted at the elements from mechanical vibration into an electrical signal (the reception signal), so that the reception signal is input to the reception circuit 117. The reception circuit 117 includes a preamplifier, an Analog-to-Digital (A/D) converter, a quadrature detection circuit, a beam former, and the like and is configured to generate reflected-wave data (reception data) by performing various types of processes on the reception signal transmitted thereto from the ultrasound probe 101. Further, the reception circuit 117 is configured to store the generated reflected-wave data into the buffer memory 120.

The pre-amplifier is configured to amplify the reception signal with respect to each of the channels and to perform a gain adjustment (a gain correction) thereon. Further, the A/D converter is configured to convert the gain-corrected reception signal into a digital signal by performing an A/D conversion on the gain-corrected reception signal. The quadrature detection circuit is configured to convert the reception signal converted to the digital signal into an In-phase signal (an I signal) and a Quadrature-phase signal (a Q signal) in a base band. Further, the beam former is configured to perform the beamforming on the IQ signals, so that data resulting from the beamforming is stored, as the reflected-wave data, into the buffer memory 120.

The reception circuit 117 is configured to generate two-dimensional reflected-wave data from two-dimensional reception signal transmitted thereto from the ultrasound probe 101. Further, the reception circuit 117 is configured to generate three-dimensional reflected-wave data from three-dimensional reception signal transmitted thereto from the ultrasound probe 101.

The buffer memory 120 is a memory configured to temporarily store therein the reflected-wave data generated by the transmission and reception circuit 110. For example, the buffer memory 120 is configured so as to be able to store therein the reflected-wave data corresponding to a predetermined number of frames. Further, when reflected-wave data corresponding to one frame is newly generated by the reception circuit 117 while the buffer memory 120 has stored therein the reflected-wave data corresponding to the predetermined number of frames, the buffer memory 120 discards reflected-wave data corresponding to one of the frames generated earliest and stores therein the newly-generated reflected-wave data corresponding to the one frame, under the control of the reception circuit 117. For example, the buffer memory 120 is realized by using a semiconductor memory element such as a Random Access Memory (RAM) or a flash memory.

The signal processing circuitry 130 is configured to read the reflected-wave data from the buffer memory 120, to perform various types of signal processing processes on the read reflected-wave data, and to output the reflected-wave data on which the various types of signal processing processes have been performed to the image generating circuitry 140 as B-mode data or Doppler data. For example, the signal processing circuitry 130 is realized by using one or more processors. The signal processing circuitry 130 is an example of a signal processing unit.

For example, every time reflected-wave data corresponding to one frame is newly stored in the buffer memory 120, the signal processing circuitry 130 is configured to read the reflected-wave data corresponding to the one frame newly stored in the buffer memory 120. After that, by performing the various types of signal processing processes on the read reflected-wave data corresponding to the one frame, the signal processing circuitry 130 is configured to newly generate B-mode data or Doppler data corresponding to the one frame. After that, every time B-mode data or Doppler data corresponding one frame is generated, the signal processing circuitry 130 is configured to output the newly-generated B-mode data or Doppler data corresponding to the one frame to the image generating circuitry 140. In the following sections, an example of the various types of signal processing processes performed by the signal processing circuitry 130 will be explained.

For example, by performing a quadrature detection, a logarithmic amplification, an envelope detection process, and the like on the reflected-wave data read from the buffer memory 120, the signal processing circuitry 130 is configured to generate B-mode data in which the signal intensity (amplitude intensity) at each sampling point is expressed as a degree of brightness. For example, the signal processing circuitry 130 is configured to output the generated B-mode data to the image generating circuitry 140.

Further, by performing a frequency analysis on the reflected-wave data read from the buffer memory 120, the signal processing circuitry 130 is configured to extract movement information of moving members (blood flows, tissues, contrast agent echo components, etc.) based on the Doppler effect from the reflected-wave data and to generate Doppler data indicating the extracted movement information. For example, the signal processing circuitry 130 is configured to generate the Doppler data indicating the extracted movement information of the moving members, by extracting, as the movement information of the moving members, an average velocity value, an average dispersion value, an average power value, and the like with respect to multiple points. The signal processing circuitry 130 is configured to output the generated Doppler data to the image generating circuitry 140.

By using the functions of the signal processing circuitry 130 described above, the ultrasound diagnosis apparatus 1B is capable of implementing a color Doppler method which may be called a Color Flow Mapping (CFM) method. According to the color flow mapping method, ultrasound wave transmission and reception is performed multiple times on a plurality of scanning lines. Further, according to the color flow mapping method, a signal (a blood flow signal) derived from a blood flow is extracted from data sequences corresponding to mutually the same position, while suppressing signals (clutter signals) derived from stationary tissues or slow-moving tissues, by applying a Moving Target Indicator (MTI) filter to the data sequences corresponding to mutually the same position. Further, according to the color flow mapping method, blood flow information, such as velocity of the blood flow, dispersion of the blood flow, power of the blood flow, and the like, is estimated on the basis of the blood flow signal. The signal processing circuitry 130 is configured to output color image data indicating the blood flow information estimated by implementing the color flow mapping method, to the image generating circuitry 140. The color image data is an example of Doppler data.

The signal processing circuitry 130 is capable of processing both types of the reflected-wave data, namely the two-dimensional reflected-wave data and the three-dimensional reflected-wave data.

The image generating circuitry 140 is configured to generate the ultrasound image data from the B-mode data or the Doppler data output from the signal processing circuitry 130. The image generating circuitry 140 is realized by using one or more processors.

For example, the image generating circuitry 140 is configured to generate two-dimensional B-mode image data in which intensities of the reflected wave are expressed with brightness levels, from two-dimensional B-mode data generated by the signal processing circuitry 130. Further, the image generating circuitry 140 is configured to generate two-dimensional Doppler image data in which the movement information or the blood flow information is visualized in an image, from two-dimensional Doppler data generated by the signal processing circuitry 130. In this situation, the two-dimensional Doppler image data obtained by visualizing the movement information in the image is velocity image data, dispersion image data, power image data, or image data combining together any of these types of image data.

In this situation, generally speaking, the image generating circuitry 140 is configured to convert (by performing a scan convert process) a scanning line signal sequence from an ultrasound scan into a scanning line signal sequence in a video format used by television, for example, and to generate display-purpose ultrasound image data. For example, the image generating circuitry 140 is configured to generate the display-purpose ultrasound image data by performing a coordinate transformation process compliant with an ultrasound scanning mode used by the ultrasound probe 101 on the data output from the signal processing circuitry 130. Further, as various types of image processing processes besides the scan convert process, the image generating circuitry 140 is configured to perform, for example, an image processing process (a smoothing process) to re-generate an average brightness value image, an image processing process (an edge enhancement process) that uses a differential filter inside an image, or the like, by using a plurality of image frames resulting from the scan convert process. Also, the image generating circuitry 140 is configured to combine text information of various types of parameters, scale graduations, body marks, and the like with the ultrasound image data.

Further, the image generating circuitry 140 is configured to generate three-dimensional B-mode image data by performing a coordinate transformation process on three-dimensional B-mode data generated by the signal processing circuitry 130. Further, the image generating circuitry 140 is configured to generate three-dimensional Doppler image data by performing a coordinate transformation process on three-dimensional Doppler data generated by the signal processing circuitry 130. In other words, the image generating circuitry 140 is configured to generate the “three-dimensional B-mode image data and three-dimensional Doppler image data” as “three-dimensional ultrasound image data (volume data)”. Further, the image generating circuitry 140 is configured to perform various types of rendering processes on the volume data, so as to generate various types of two-dimensional image data used for displaying the volume data on the display 103.

Examples of the rendering processes performed by the image generating circuitry 140 include a process of generating Multi Planar Reconstruction (MPR) image data from the volume data by using a Multi Planar Reconstruction (MPR) method. Further, other examples of the rendering processes performed by the image generating circuitry 140 include a Volume Rendering (VR) process by which two-dimensional image data reflecting three-dimensional information is generated. The image generating circuitry 140 is an example of an image generating unit.

The B-mode data and the Doppler data are each ultrasound image data before the scan convert process. The data generated by the image generating circuitry 140 is the display-purpose ultrasound image data after the scan convert process. The B-mode data and the Doppler data may be referred to as raw data.

The image memory 150 is a memory configured to store therein various types of image data generated by the image generating circuitry 140. Further, the image memory 150 is also configured to store therein the data generated by the signal processing circuitry 130. An operator is able to invoke the B-mode data and the Doppler data stored in the image memory 150 after a diagnosis process, for example. The invoked data serves as display-purpose ultrasound image data after being routed through the image generating circuitry 140. For example, the image memory 150 is realized by using a semiconductor memory element such as a Random Access Memory (RAM) or a flash memory, or a hard disk, an optical disk, or the like.

The storage circuit 160 is configured to store therein control programs for performing the scan (transmitting and receiving ultrasound waves), image processing processes, and display processes, as well as various types of data such as diagnosis information (e.g., patient IDs, medical doctors' observations, etc.), diagnosis protocols, and various types of body marks. Further, the storage circuit 160 may also be used for saving any of the data stored in the image memory 150, as necessary. For example, the storage circuit 160 is realized by using a semiconductor memory element such as a flash memory, or a hard disk, an optical disk, or the like.

The controlling circuitry 170 is configured to control the entirety of processes performed by the ultrasound diagnosis apparatus 1B. More specifically, the controlling circuitry 170 is configured to control processes performed by the transmission circuit 116, the reception circuit 117, the signal processing circuitry 130, and the image generating circuitry 140, on the basis of the various types of setting requests input from the operator via the input device 102 and the various types of control programs and various types of data read from the storage circuit 160. Further, the controlling circuitry 170 is configured to control the display 103 so as to display ultrasound images based on the display-purpose ultrasound image data stored in the image memory 150. For example, the controlling circuitry 170 is configured to control the display 103 so as to display a B-mode image based on B-mode image data or a color image based on color image data. Further, the controlling circuitry 170 is configured to control the display 103 so as to display a color image superimposed on a B-mode image. The controlling circuitry 170 is an example of a display controlling unit or a controlling unit. For example, the controlling circuitry 170 is realized by using one or more processors. The ultrasound images are examples of images.

Further, the controlling circuitry 170 is configured to control the ultrasound scan, by controlling the ultrasound probe 101 via the transmission and reception circuit 110.

The processing circuitry 180 includes a lesion detection function 180a, a frame result acquisition function 180b, a threshold update function 180c, and a learning function 180d. For example, each of the processing functions of the lesion detection function 180a, the frame result acquisition function 180b, the threshold update function 180c, and the learning function 180d, which are components of the processing circuitry 180 illustrated in FIG. 9, is recorded in the storage circuit (memory) 160 in the form of a computer-executable program. The processing circuitry 180 reads each program from the memory 160 and executes each read program to implement the function corresponding to each program. In other words, the processing circuitry 180 in a state of reading each program has each function illustrated in the processing circuitry 180 of FIG. 9. The processing circuitry 180 is implemented by, for example, a processor.

The processing circuitry 180 executes the same processing as the processing executed by the processing circuitry 11 according to the first embodiment or the processing circuitry 11A according to the second embodiment on various images (videos) stored in the image memory 150, and causes the display 103 to display the processing result.

The lesion detection function 180a has the same function as that of the lesion detection function 111 according to the first embodiment or the second embodiment. The frame result acquisition function 180b has the same function as the frame result acquisition function 112 according to the first embodiment or the second embodiment. The threshold update function 180c has the same function as the threshold update function 113 according to the first embodiment or the second embodiment. The learning function 180d has the same function as the learning function 115 according to the second embodiment. Therefore, according to the ultrasound diagnosis apparatus 1B according to the third embodiment, the same effects as those of the first embodiment or the second embodiment are obtained.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). One or more processors are configured to realize the functions by reading the programs saved in the storage circuit 160 and executing the read programs. Alternatively, instead of having the programs saved in the storage circuit 160, it is also acceptable to directly incorporate the programs into the circuits of the one or more processors. In that situation, the one or more processors are configured to realize the functions by reading and executing the programs incorporated in the circuits thereof. Further, the processors according to the present embodiments do not each necessarily have to be structured as a single circuit. It is also acceptable to structure one processor by combining together a plurality of independent circuits, so as to realize the functions thereof. Further, two or more of the pieces of circuitry (e.g., the signal processing circuitry 130, the image generating circuitry 140, the controlling circuitry 170, and the processing circuitry 180) illustrated in FIG. 9 may be integrated in a single processor so as to realize the functions thereof. In other words, the signal processing circuitry 130, the image generating circuitry 140, the controlling circuitry 170, and the processing circuitry 180 may be integrated into one piece of processing circuitry realized by the processor.

An overall configuration of the ultrasound diagnosis apparatus 1B according to the third embodiment has thus been explained. According to the ultrasound diagnosis apparatus 1B according to the third embodiment, the same effects as those of the first embodiment or the second embodiment are obtained.

According to the lesion detection apparatus, the lesion detection method, and the ultrasound diagnosis apparatus according to each embodiment, it is possible to determine the lesion type in the current frame based on the detection results in the previous frames, appropriately update the detection threshold according to the lesion type, and improve the lesion detection accuracy.

As compared with a case where the fixed detection threshold is set, it is possible to provide different detection thresholds by separating the lesion type in the current frame from the lesion type other than the lesion type in the current frame. As a result, it is possible to reduce the possibility of detecting the lesion that does not exist in the current frame, reduce the number of false detection frames, and further improve the lesion detection accuracy.

In addition, since the number of detection frames (false detection frames) of the lesion type other than the lesion type in the current frame is reduced, it is possible to improve visual experience of the user and reduce interference.

In addition, since the possibility of detecting the lesion not existing in the current frame is reduced while the possibility of detecting the lesion existing in the current frame is increased, it is possible to improve the lesion detection accuracy without increasing the total number of detection frames.

In addition, the lesion detection apparatus, the lesion detection method, and the ultrasound diagnosis apparatus according to each embodiment can be applied to the detection of a plurality of types of lesions in various images using various existing detection models.

In the above-described embodiment, the processing circuitry is not limited to one implemented by a single processor, and may be configured by combining a plurality of independent processors, and each processor may implement each processing function by executing the program. In addition, each processing function of the processing circuitry may be implemented by being appropriately distributed or integrated into a single or a plurality of processing circuits. In addition, each processing function of the processing circuitry may be implemented by a mixture of hardware such as circuitry and software. Furthermore, here, an example of a case has been described where the program corresponding to each processing function is stored in a single storage circuit, but the embodiment is not limited thereto. For example, the program corresponding to each processing function may be distributed and stored in a plurality of storage circuits, and the processing circuitry may read and execute each program from each storage circuit.

Furthermore, in each of the above-described embodiments, an example has been described in which each functional unit in the present specification is implemented by the processing circuitry, but the embodiment is not limited thereto. For example, each functional unit in the present specification may be implemented by the processing circuitry described in the embodiment, and the same function may be implemented by only hardware, only software, or a mixture of hardware and software.

In addition, in the above-described embodiment, each component of each apparatus illustrated in the drawings is functionally conceptual, and does not necessarily need to be physically configured as illustrated in the drawings. That is, a specific form of the distribution or integration of each apparatus is not limited to the illustrated one, and all or a part thereof can be functionally or physically distributed or integrated in an arbitrary unit according to various loads, usage conditions, and the like. Furthermore, all or an arbitrary part of each processing function performed in each apparatus can be implemented by the CPU and a program analyzed and executed by the CPU, or can be implemented as hardware based on wired logic.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
processing circuitry that detects a lesion based on a detection threshold for detecting a lesion corresponding to a lesion type in each of a plurality of imaged frames,
acquires detection results of the lesion in the plurality of frames, and
determines a lesion type based on at least one of continuity and appearance frequency of the lesion type in the detection results of the plurality of frames, and updates the detection threshold according to the determined lesion type,
wherein the processing circuitry detects the lesion based on the updated detection threshold.

2. The ultrasound diagnosis apparatus according to claim 1, wherein
the processing circuitry acquires detection results of the lesion in a plurality of previous frames before a current frame,
determines the lesion type in the current frame based on at least one of continuity and appearance frequency of the lesion type in the detection results of the plurality of previous frames, and updates the detection threshold according to the determined lesion type in the current frame, and
detects the lesion in the current frame based on the updated detection threshold.

3. The ultrasound diagnosis apparatus according to claim 2, wherein the processing circuitry further determines the lesion type in the current frame based on the confidence of the lesion type in the detection results of the previous frames.

4. The ultrasound diagnosis apparatus according to claim 3, wherein the processing circuitry updates the detection threshold so as to lower the detection threshold in a case where at least one of the continuity and the appearance frequency, and the confidence satisfy a preset condition.

5. The ultrasound diagnosis apparatus according to claim 4, wherein the processing circuitry sets a lower limit to the updated detection threshold.

6. The ultrasound diagnosis apparatus according to claim 3, wherein the processing circuitry updates the detection threshold so as to raise the detection threshold in a case where at least one of the continuity and the appearance frequency, and the confidence satisfy a preset condition.

7. The ultrasound diagnosis apparatus according to claim 6, wherein the processing circuitry sets an upper limit to the updated detection threshold.

8. The ultrasound diagnosis apparatus according to claim 1, wherein the processing circuitry causes a display to display a detection frame indicating the detected lesion in a case where the lesion is detected based on the updated detection threshold.

9. The ultrasound diagnosis apparatus according to claim 8, wherein the processing circuitry causes the display to display the detection frame so as to change a color of the detection frame according to the detected lesion type.

10. The ultrasound diagnosis apparatus according to claim 8, wherein the processing circuitry causes the display to display the detection frame together with confidence of each of the detected lesion type.

11. The ultrasound diagnosis apparatus according to claim 8, wherein the processing circuitry causes the display to display information on the update in a case where the detection threshold is updated.

12. The ultrasound diagnosis apparatus according to claim 1, wherein the processing circuitry updates the detection threshold in the current frame using a detection threshold output from a learned model by inputting the detection results in the previous frames to the learned model learned so as to output the detection threshold in the current frame based on the detection results in the previous frames.

13. A lesion detection apparatus comprising:
processing circuitry that detects a lesion based on a detection threshold for detecting a lesion corresponding to a lesion type in each of a plurality of imaged frames,
acquires detection results of the lesion in the plurality of frames, and
determines a lesion type based on at least one of continuity and appearance frequency of the lesion type in the detection results of the plurality of frames, and updates the detection threshold according to the determined lesion type,
wherein the processing circuitry detects the lesion based on the updated detection threshold.

14. A lesion detection method comprising:
a lesion detection step of detecting a lesion based on a detection threshold for detecting a lesion corresponding to a lesion type in each of a plurality of imaged frames;
a frame result acquisition step of acquiring detection results of the lesion in the plurality of frames; and
a threshold update step of determining a lesion type based on at least one of continuity and appearance frequency of the lesion type in the detection results of the plurality of frames, and updating the detection threshold according to the determined lesion type,
wherein the lesion is detected based on the updated detection threshold in the lesion detection step.
